(19) United States
(12) Patent Application Publication
(10) Pub. No.: US 2009/0177133 A1
(43) Pub. Date: Jul. 9, 2009

(54) Reduced pressure dressing coated with biomolecules

(76) Inventors: Kristine Kieswetter, San Antonio, TX (US); Amy McNulty, San Antonio, TX (US)

Correspondence Address: KINETIC CONCEPTS, INC.;C/O SONNENSCHEIN NATH & ROSENTHAL LLP, P.O. BOX 061080, WACKER DRIVE STATION, SEARS TOWER, CHICAGO, IL, 60606, US

(21) Appl. No.: 12/006,721

(22) Filed: Jan. 4, 2008

(51) Int. Cl. A61F 13/00; A61L 15/00

(52) U.S. Cl. 602/48; 602/75

(57) ABSTRACT

A reduced pressure dressing coated with biomolecules including a polymer material layer and at least one biomolecule selected from the group consisting of a hemostatic agent, an antioxidant agent, and a nitric oxide promoter, the at least one biomolecule absorbed into a portion of the polymer material layer. The present reduced pressure dressing coated with biomolecules further includes methods for making same.

### BACKGROUND OF THE INVENTION

1. Field of the Invention

The present invention relates generally to reduced pressure dressings, and more particularly to reduced pressure dressings coated with biomolecules.

2. Description of Related Art

Chronic wounds continue to be problematic. There are over 7 million chronic wounds in the United States. The mean hospital charge for one of these types of wounds (pressure ulcers) has been estimated at over $20,000. Besides the monetary cost associated with healing chronic wounds, these wounds may be debilitating, affecting the quality of life for those afflicted.

Currently, there is no single treatment for chronic wounds that is effective in all cases. Rather, treatment for chronic wounds is not highly advanced. Typically, a physician will prescribe a certain treatment protocol and if no significant improvement is experienced within a few weeks, then another treatment protocol is prescribed. This process continues until the wound heals or until no further treatment protocols are available. People may endure these chronic wounds for years. Recent medical developments have improved the treatment of chronic wounds by the use of reduced pressure systems, which employ manifolds or systems that directly contact the tissue site and distribute reduced pressure to the tissue site.

One of the challenges to using these protocols is the instability of the tissue site. For example, a tissue site that is bleeding or oozing a fluid may be problematic for the use of such manifold systems. This is because the scaffolds and dressings of these manifold systems directly contact the tissue site, thus they further irritate the tissue site causing additional inflammation.

Further, tissue sites are typically very hostile environments to topically applied biomolecules. This is because the tissue sites contain a large number of proteases and as soon as a biomolecule is placed directly on a tissue site the proteases degrade the biomolecule. In addition, with particular chronic diseases, such as diabetes related wounds, it has been found that the tissue sites do not vasodilate very well, so blood flow is impeded.

Also, when topical antimicrobial coatings, such as silver nitrate and sulfadiazine, are applied to conventional dressings, data shows that the release of the ointment to the tissue site occurs for about the first 30 minutes after application, and that very little ointment is released after that period. The availability of the ointment beyond its initial application is substantially limited. This may be due to the fact that most topical antimicrobial coatings are not bound or bonded to the dressing, but just applied as a thin layer. Thus, there is no time delivery functionality associated with these conventional dressings with topical antimicrobial coatings applied to their dressing surface.

Another challenge related to reduced pressure manifold type systems is that the reduced pressure causes the foam dressings and/or scaffolds associated with these systems to compress into the underlying tissue. This pressure further pulls some of the tissue site tissue up into the cells, pores, voids, and apertures of the dressings and scaffolds. Thus, any topical application to a foam dressing of a manifold will not react with the tissue that is pulled into the cells, pores, voids, and apertures of these types of manifold foam dressings.

Additionally, these types of systems engender a fluid flow gradient that facilitates the flow of exudate away from the tissue site. Thus, the fluids associated with a tissue site, such as an exudate, are flowing away from the tissue site not towards it. Thus, any topical application of biomolecules applied directly to the tissue site prior to sealing the tissue site with a dressing or scaffold, would also flow away from the tissue site with the exudate that is being evacuated during such treatment.

### BRIEF SUMMARY OF THE INVENTION

The problems presented with these conventional chronic wound treatment protocols using biomolecules are solved by an improved reduced pressure dressing coated with biomolecules. The biomolecule dressing, when used with a reduced pressure therapy, decreases the magnitude of degradation to the biomolecule(s) caused by the proteases associated with tissue sites. In one exemplary embodiment, the biomolecule dressing contains nitric oxide that improves the blood flow in wounds, such as diabetes related tissue sites.

In another exemplary embodiment, a biomolecule dressing provides the time release of biomolecules to a tissue site over a preferable period of time. The polymer layer of the biomolecule dressing may be derivatized so that it may bond to certain biomolecules for improved time release to the tissue site.

In still another exemplary embodiment, a biomolecule dressing further improves the hemostasis of a tissue site prior to application of reduced pressure therapy. The biomolecule dressing decreases the amount of excessive interspatial fluid or potential bleeding out prior to the application of the reduced pressure.

In another exemplary embodiment, a reduced pressure dressing coated with biomolecules includes a polymer material layer and at least one biomolecule selected from the group consisting of a hemostatic agent, an antioxidant agent, and a nitric oxide promoter, the at least one biomolecule absorbed into a portion of the polymer material layer. The reduced pressure dressing coated with biomolecules further includes methods for making same.

Other objects, features, and advantages of the present invention will become apparent with reference to the drawings and detailed description that follow. In the drawings, like or similar elements are designated with identical reference numerals throughout the several views and figures thereof, and various depicted elements may not be drawn necessarily to scale.

### BRIEF DESCRIPTION OF THE DRAWINGS

A more complete understanding of the method and apparatus of the present invention may be obtained by reference to the following Detailed Description when taken in conjunction with the accompanying Drawings wherein:

FIG. 1 illustrates a perspective view of a biomolecule dressing according to an embodiment of the present invention;

FIG. 2 illustrates a cross-sectional view of the biomolecule dressing along lines 2-2 of FIG. 1 according to an embodiment of the present invention;

FIG. 3 illustrates a cross-sectional view of a biomolecule dressing having an additional outside layer of biomolecules according to another embodiment of the present invention;

FIG. 4 illustrates a cross-sectional view of a biomolecule dressing having several additional outside layers of biomolecules according to another embodiment of the present invention;

FIG. 5 illustrates a cross-sectional view of a biomolecule dressing NPWT apparatus according to an embodiment of the present invention;

FIG. 6 illustrates an interface of a tissue site and a biomolecule dressing including an exemplary biomolecule according to an embodiment of the present invention;

FIG. 7 illustrates a cross-sectional view of a biomolecule dressing having different biomolecules absorbed in the polymer layer according to another embodiment of the present invention;

FIG. 8 illustrates a plot depicting the interstitial pressure gradient for different magnitudes of reduced pressure applied and their corresponding magnitude of reduced pressure measured at certain depths of a tissue site;

FIG. 9 illustrates a flow chart of an exemplary process for making a biomolecule dressing according to an embodiment of the present invention; and

FIG. 10 illustrates a flow chart of an exemplary process for making a biomolecule dressing according to another embodiment of the present invention.

### DETAILED DESCRIPTION OF THE PREFERRED EMBODIMENT

In the following detailed description of the preferred embodiments, reference is made to the accompanying drawings that form a part hereof, and in which is shown by way of illustration specific preferred embodiments in which the invention may be practiced. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present invention is defined only by the appended claims.

As used herein, the term “bioresorbable” generally means a material that slowly dissolve and/or digest in a living being, such as a human, and may be synonymous with bioabsorbable, biodissolvable, biodegradable, and the like. Bioresorbable describes the property of a material to break down when the material is exposed to conditions that are typical of those present in a wound bed into degradation products that can be removed from the tissue site within a period that roughly coincides with the period of wound healing. Such degradation products can be absorbed into the body of the patient or can be transmitted into another layer of the dressing. The period of wound healing is to be understood to be the period of time measured from the application of a dressing to the time that the wound is substantially healed. This period can range from a period of several days for simple skin abrasions on rapidly healing patients, to several months for chronic wounds on patients that heal more slowly. It is intended that the subject dressing can be fabricated so that the time required for bioresorption and/or bioabsorption of the scaffold material can be tailored to match the type of wound and the time necessary for healing. For example, in some dressings of the subject invention, the scaffold material may be designed to degrade within a period of one week, while in other dressings it may be designed to degrade within a period of one-to-three months, or even longer if desirable.

The term “reduced pressure” as used herein generally refers to a pressure less than the ambient pressure at a tissue site that is being subjected to treatment. In most cases, this reduced pressure will be less than the atmospheric pressure at which the patient is located. Alternatively, the reduced pressure may be less than a hydrostatic pressure of tissue at the tissue site. Although the terms “vacuum” and “negative pressure” may be used to describe the pressure applied to the tissue site, the actual pressure applied to the tissue site may be significantly less than the pressure normally associated with a complete vacuum. Reduced pressure may initially generate fluid flow in the tube and the area of the tissue site. As the hydrostatic pressure around the tissue site approaches the desired reduced pressure, the flow may subside, and the reduced pressure is then maintained. Unless otherwise indicated, values of pressure stated herein are gauge pressures.

The term “tissue site” as used herein refers to a wound or defect located on or within any tissue, including but not limited to, bone tissue, adipose tissue, muscle tissue, dermal tissue, vascular tissue, connective tissue, cartilage, tendons, or ligaments. The term “tissue site” may further refer to areas of any tissue that are not necessarily wounded or defective, but are instead areas in which it is desired to add or promote the growth of additional tissue. For example, reduced pressure tissue treatment may be used in certain tissue areas to grow additional tissue that may be harvested and transplanted to another tissue location.

The biomolecule dressing may be used on different types of wounds or tissues, such as surface wounds, deep-tissue wounds, and percutaneous wounds. For example, the biomolecule dressing may be placed adjacent to a bone of a patient and then the skin of the patient may be closed.

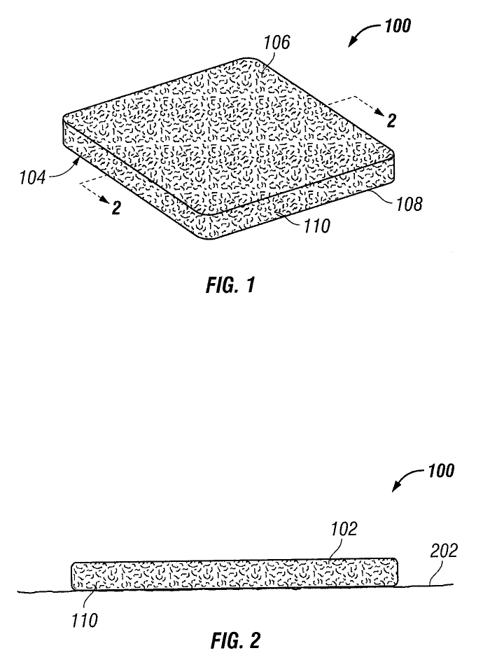

Referring to FIGS. 1 and 2, a biomolecule dressing 100 is illustrated. In this embodiment, the biomolecule dressing 100 is a polymer layer that includes a bottom surface 104, top surface 106, and sides 108 that join bottom surface 104 to top surface 106. FIG. 2 illustrates a cross-sectional view of the biomolecule dressing 102 along the lines 2-2 of FIG. 1 and is shown adjacent to a tissue site 202. Typically, the bottom surface 104 of the body 102 substantially contacts and/or is adjacent to tissue site 202. Biomolecule dressing 100 further includes flow channels 110 for allowing exudates and liquids to flow through the biomolecule dressing. Biomolecule dressing 100 may be coated partially or completely with a desired biomolecule as described herein.

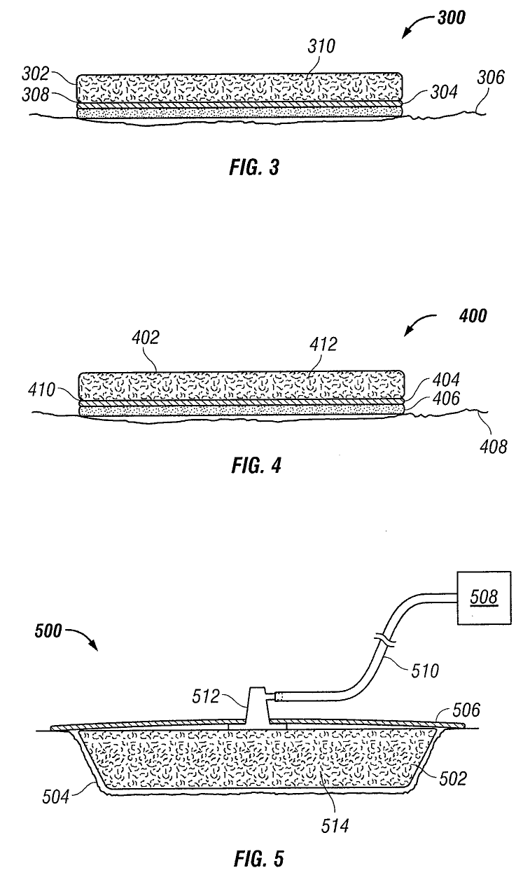

Referring to FIG. 3, a biomolecule dressing 300 according to an exemplary embodiment of the invention includes a polymer layer 302 having an additional layer of biomolecules 304 applied to a bottom surface 308 of the polymer layer 302. In this embodiment, the additional layer of biomolecules 304 substantially contacts and/or is adjacent to a tissue site 306. The polymer layer 302 may have biomolecules absorbed and/or adsorbed onto or through the polymer layer 302. The additional layer of biomolecules 304 may be chemically bound to the bottom surface 308 of the polymer layer 302. In another embodiment, the additional layer of biomolecules 304 may be applied to the polymer layer and held in place by surface tension, ionic bonds, covalent bonds, or Van der Waals forces. These bonds and forces are achieved through the chemistry of the biomolecules of the additional layer of biomolecules 304 and the polymer layer 302.

The biomolecules of the additional layer of biomolecules 304 and the polymer layer 302 may be the same or different biomolecules. For example, the polymer layer 302 may be coated partially or completely throughout with an antioxidant and the additional layer of biomolecules 304 may also be an antioxidant layer of material. In another example, the polymer layer 302 may be coated partially or completely throughout with an antioxidant, while the additional layer of biomolecules 304 may be a different hemostatic agent, such as poly-N-acetyl-glucosamine (“GlcNAc”). Any combination of biomolecules may be used with the biomolecule dressing 300. Biomolecule dressing 300 further includes flow channels 310 for allowing exudates and liquids to flow through the biomolecule dressing.

Referring to FIG. 4, a biomolecule dressing 400 according to an exemplary embodiment of the invention includes several additional layers of material located adjacent to a bottom surface 410 of a polymer layer 402 of the biomolecule dressing 400. An inert layer 404 is interspersed between the bottom surface 410 of the polymer layer and an additional layer of biomolecules 406. In this embodiment, the additional layer of biomolecules 406 substantially contacts and/or is adjacent to the tissue site 408. In this embodiment, the additional layer of biomolecules 406 may consist of the same or different biomolecules as contained in the polymer layer 402. The inert layer 404 may be used to provide a time release element to the biomolecule dressing 400 by providing a layer of material that does not provide a hemostatic effect but that is bioresorbed, biorecycled, dissolved, or the like over time prior to the polymer layer 406 coming in direct contact with the tissue site 408 for further hemostatic effect. Biomolecule dressing 400 further includes flow channels 412.

In another embodiment, the biomolecule dressing may include any number of inert layers or additional layers of biomolecules in addition to the polymer layer. These inert layers and/or additional layers of biomolecules may be alternating layers of adjacent common layers. Further, they may be of different types of biomolecules or the same biomolecules as other or adjacent layers of the biomolecule dressing. Additionally, the biomolecule dressings described herein may include embodiments of a reduced pressure treatment system.

Referring to FIG. 5, a reduced pressure treatment system 500 according to an exemplary embodiment of the invention includes a biomolecule dressing 502 for insertion substantially on top of a tissue site 504 and a wound drape 506 for sealing enclosure of the biomolecule dressing 502 and the tissue site 504. As shown, biomolecule dressing 502 includes a polymer layer that includes a biomolecule absorbed throughout the polymer layer. After placement of the biomolecule dressing 502 at the tissue site 504 and sealing with the drape 506, the biomolecule dressing 502 is placed in fluid communication with a vacuum pump or reduced pressure source 508 for promotion of reduced pressure treatment and fluid drainage. A reduced pressure delivery tube 510 allows fluid communication between the reduced pressure source 508 and a tubing connector 512 that is in fluid communication with the biomolecule dressing 500. The tubing connector 512 is located typically between the biomolecule dressing 502 and the drape 506 and extends through a portion of the drape 506. Drainage is facilitated by flow channels 514 located in the biomolecule dressing 502.

The biomolecule dressing 502 is preferably placed in fluid communication via the connector 512 and the reduced pressure delivery tube 510, with the reduced pressure source 508. The drape 506, which preferably comprises an elastomeric material at least peripherally covered with a pressure sensitive, acrylic adhesive, is positioned over the biomolecule dressing 502 to substantially seal the biomolecule dressing 502 at the tissue site 504. As shown in FIG. 5, the biomolecule dressing 502 substantially contacts and/or is adjacent to the tissue site 504. In this embodiment, the biomolecule dressing 502 conforms well to uneven surfaces, such as deep wound bodies and the like.

In another embodiment, any of the other biomolecule dressings 100, 300, and 400 may be used with the reduced pressure treatment system 500 shown in FIG. 5 in place of or in addition to biomolecule dressing 502. In yet another embodiment, the order of the layers of the biomolecules and inert layers as described herein may be arranged in any order desired.

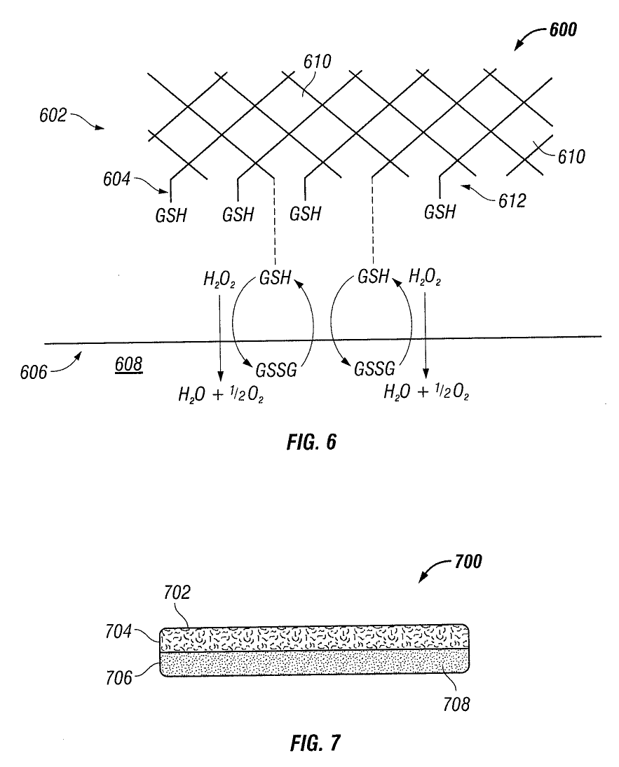

FIG. 6 illustrates an interface 606 of a tissue site 608 and a biomolecule dressing 600 including an exemplary antioxidant biomolecule, reduced glutathione (“GSH”), located at or near the interface 606. GSH is an antioxidant that is bound to a polymer layer 602 of the biomolecule dressing 600. The GSH contacts the tissue site 608 and some of the GSH is released when it contacts a reactive species in the tissue site 608, such as hydrogen peroxide or oxygen. Hydrogen peroxide is a weak acid that possesses strong oxidizing properties. Here the hydrogen peroxide is reduced to water and oxygen and, in the presence of GSH, oxidizes the GSH to oxidized glutathione (“GSSG”) via glutathione reductase. As is shown, the glutathione oxidation reduction cycle provides a ready source of GSH for use in improving the hemostasis of the tissue site 608 by removing harmful oxygen free radicals (“oxygen radicals” and/or “oxyradicals”).

In this embodiment, the polymer layer 602 of the biomolecule dressing 600 depicts the polymer layer 602 slightly enlarged to show the reticulated open cells 610 of the polymer layer 602. In this embodiment, the GSH is located at an outer surface 612 of the polymer layer 602. In addition, GSH is further located throughout the reticulated open cells 610 of the polymer layer 602.

Referring to FIG. 7, a biomolecule dressing 700 according to an exemplary embodiment of the invention includes two different biomolecules absorbed and/or adsorbed within a polymer layer 702 of the biomolecule dressing 700. In this embodiment, a first portion 704 of the polymer layer 702 may include biomolecules that are different than the biomolecules of a second portion 706 of the polymer layer 702. In another embodiment, the first portion 704 of the polymer layer 702 may include biomolecules that are similar to the biomolecules of the second portion 706, but in a different concentration. Additionally, a further embodiment may include additional portions of biomolecules that are different or similar and in substantially the same or different concentrations than those in the other portions of the polymer layer 702. Further, polymer layer 702 may include flow channels 708.

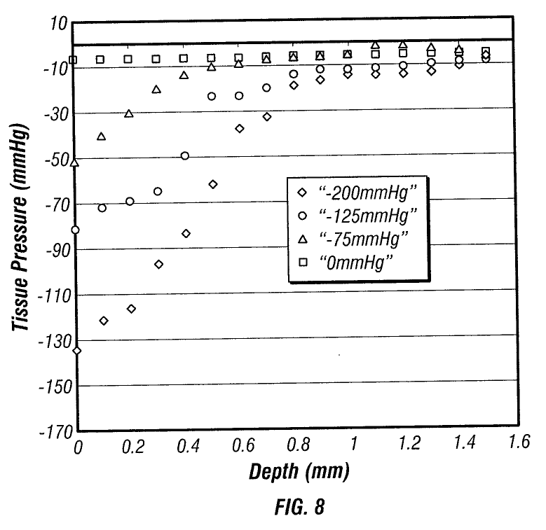

FIG. 8 illustrates a plot depicting the measured tissue pressure at various depths within a tissue site for various applied magnitudes of reduced pressure. For example, the “diamond” plot represents an applied reduced pressure having a magnitude of −200 mm Hg. In this example, a reduced pressure applied at −200 mm Hg results in a measured tissue pressure of approximately −135 mm Hg immediately below the surface (fluid side) of the tissue site. Similarly, at a depth of about 1 mm in the tissue site the measured reduced pressure is approximately −15 mm Hg. In one embodiment, the biomolecule dressing is used with a reduced pressure system that applies a reduced pressure to the tissue site. The reduced pressure slightly compresses the polymer layer while concurrently pulling the tissue at the tissue site into the cells, pores, voids, and apertures of the polymer layer. Because the biomolecules are located throughout the polymer layer, they remain in contact with the tissue that is being brought into the polymer layer for improved hemostasis as described herein.

Additionally, the pressure gradient created by the reduced pressure treatment system causes a fluid flow from the tissue site through the pores, voids, and apertures of the polymer layer. Nevertheless, the fluid flow away from the tissue site is still in contact with the biomolecules as it travels through the pores, voids, and apertures of the polymer layer, thus providing for improved hemostasis and healing during reduced pressure treatment.

In one embodiment, a biomolecule may be a hemostatic agent, such as GlcNAc. Some other exemplary hemostatic agents may include without limitation thrombin, fibrinogen, or fibrin constituted in an aqueous solution of a non-acidic, water-soluble or water-swellable polymer, including but not limited to methyl cellulose, hydroxyalkyl cellulose, keratin sulfate, water-soluble chitosan, N-acetyllactosamine synthase, salts of carboxymethyl carboxyethyl cellulose, chitin, salts of hyaluronic acid, alginate, propylene glycol alginate, glycogen, dextran, carrageenans, chitosan, starch, amylose, and the aldehyde-oxidized derivatives thereof. The hemostatic agent may be applied as a thin layer on a surface of the polymer layer or it may be absorbed and/or adsorbed throughout the entire polymer layer as described herein.

In another embodiment, a biomolecule may be an antioxidant. Some exemplary antioxidants include without limitation glutathione, lipoic acid, vitamin E, ascorbic acid, trolox, tocopherols, and tocotrienols. Antioxidants may promote healing of the tissue site by protection of fibroblasts and keratinocytes against destruction by inflammatory mediators, such as free radicals. These highly reactive substances in the tissue site will damage or destroy key cell components (e.g. membranes and DNA) rapidly if they are not removed or neutralized. Typically, oxyradicals are generated in the many thousand mitochondria located inside each cell, where nutrients like glucose are burned using oxygen to make energy. In addition, some antioxidants, such as glutathione, recycle other well-known antioxidants such as vitamin C and vitamin E, keeping them in their active state for improved hemostatic conditions of the tissue site. Antioxidants, such as vitamin E are particularly effective in hemostasis and healing of wounds in diabetes related chronic wounds.

In one embodiment, the biomolecule dressing delivers the antioxidant into the tissue site during reduced pressure treatment and enhances the effectiveness of the therapy. In another embodiment, the antioxidant may be applied as a thin layer on a surface of the polymer layer or it may be absorbed and/or adsorbed throughout the entire polymer layer as described herein. In one embodiment, the antioxidant that is contained in a polymer layer is glutathione, lipoic acid, and/or vitamin E. Additional layers of this antioxidant may be further applied to a surface of the polymer layer of the biomolecule dressing.

In another embodiment, a biomolecule may be nitric oxide, nitric oxide donor compounds, nitric oxide precursor compounds, and/or upregulators of nitric oxide compounds. The contact of nitric oxide improves the blood flow at diabetic-related tissue sites, for example, thus, improving vasodilation at the tissue site. Further, adequate rates of nitric oxide production are necessary for intact wound healing, thus nitric oxide further improves the hemostasis of a tissue site. The biomolecule dressing improves healing of tissue sites by mediating such processes as angiogenesis. Angiogenesis is the process of new blood vessel growth from preexisting vessels that include several steps, such as dissolution of basement endothelial cells, endothelial cell migration, adhesion, proliferation, and tube differentiation. An exemplary nitric oxide precursors is L-arginine. One example of a nitric oxide upregulator is nitric oxide synthase. One example of a nitric oxide donor is nitroprusside.

In one embodiment, the biomolecule dressing may further include a delivery agent for delivering the biomolecules from the polymer layer to the tissue site. Some exemplary delivery agents are lipisomes, microspheres, dextran, hyaluronic acid, glycoamino glycans (“GAGs”), and starches. In one embodiment, the delivery agent is bound to the polymer layer first and then the desired biomolecule is bound to the delivery agent in a separate reaction. In another embodiment, the delivery agent and desired biomolecule is bound to the polymer layer in one reaction.

In yet another embodiment, a spike coating is applied to the polymer layer of the biomolecule dressing that may be activated by an ion beam that drives the molecules off of the polymer layer. Further, additional layers of biomolecules may be applied on the polymer layer of the biomolecule dressing and released in this manner to provide additional time release delivery of such biomolecules.

In another embodiment, the biomolecule dressing may include chemically reacting the polymer layer with the biomolecules, such as derivatizing the polymer layer prior to contacting it with the biomolecules. For example, polyurethane esters have ester linkages that can be derivatized, which provides a reaction site for the N-acetyl-glucosamine and either ionically or covalently bond it to the ester linkage. The biomolecule dressing may include chemically modifying the polymer layer to bond ionically or covalently with the biomolecules. For example, if a greater period of time release is desired, the N-acetyl-glucosamine may be ionically bonded to the polyurethane ester rather than covalently bonded. The complete and direct contact of the ionically bonded biomolecules provides for improved time release functionality. For example, silver may be applied to the polymer layer in a metallic form, and when exposed to the tissue site the silver becomes positively charged. When it contacts the extracellular matrix of the tissue site, which is highly negative charge, the silver becomes bonded to the extracellular matrix of the tissue site.

In one embodiment, the polymer layer is a polymer-type material that is capable of acting as a manifold for providing reduced pressure to the tissue site. Further, the polymer layer may include binding sites for the biomolecules. In general, the polymer layer may be a foam or other 3-dimensional porous structure suitable for use in applications as herein described. Some exemplary polymer layer materials include GranuFoam® and WhiteFoam™ that are manufactured by KCl of San Antonio, Tex. Some additional exemplary polymer layer materials include without limitation polyurethane, cellulose, carboxylated butadiene-styrene rubber, polyester foams, hydrophilic epoxy foams, polyacrylate, PVC, and polyethylene (“PE”). The polymer layer may be selected to deliver appropriate amounts of biomolecule to the tissue site over time.

In one embodiment, the biomolecule dressing may be used as a reduced pressure manifold, or the biomolecule dressing may be used as a non-manifold type dressings, foams, or polymer-type materials. In another embodiment, the biomolecule dressing may serve as a conventional or bioresorbable scaffold. In one aspect, the polymer layer of the biomolecule dressing may be bioresorbable, thus not requiring replacement or removal from the tissue site.

In one embodiment, the polymer layer may be made of bioresorbable material, including polymer-type materials. Typically, these bioresobrable materials are broken down or metabolized by the body of a patient to smaller components that may ultimately be released from the body. The bioresorbable material may be chosen for its strength over a period of time to allow tissue to regenerate before the material is bioresorbed. For example, the bioresorbable material may include without limitation polylactide (“PLA”) (both L-lactide and D,L-lactide), copolymer of Poly(L-lactide-co-D,L-lactide), polyglycolic acid (“PGA”), alpha esters, saturated esters, unsaturated esters, orthoesters, carbonates, anhydrides, ethers, amides, saccharides, polyesters, polycarbonates, polycaprolactone (“PCL”), polytrimethylene carbonate (“PTMC”), polydioxanone (“PDO”), polyhydroxybutyrate, polyhydroxyvalerate, polydioxanone, polyorthoesters, polyphosphazenes, polyurethanes, collagen, hyaluronic acid, chitosan, polymers incorporating one or more of hydroxyapatite, coralline apatite, calcium phosphate, calcium sulfate, calcium sulfate, calcium carbonate, carbonates, bioglass, allografts, autografts, and mixtures and/or co-polymers of these compounds. These compounds may be combined to produce co-polymers with fixed ratios of the polymers, such as 70:30 ratio of L-lactide-co-D,L-lactide. In addition, these compounds, polymers, and co-polymers may be linear or non-linear compounds.

As described above, inert layers may be interposed between layers of the biomolecules within and on the polymer layer of the biomolecule dressing. For example, several layers alternating between biomolecules and inert layers may be applied or chemically bonded to the polymer layer for improved time release of the biomolecules during the period that the biomolecule dressing is applied to the tissue site. This way a desired amount of the biomolecules is delivered over a course of a therapy and not all at once as is found with conventional dressings containing topical antimicrobial coatings.

In one embodiment, the biomolecule dressing includes dip coating the polymer layer into the biomolecules of a desired application. In another embodiment, a spraying or pressure treating operation may incorporate the biomolecules into the polymer layer.

Flow channels 110, 310, 514, and 708 allow distribution of reduced pressure to and/or transportation of exudates from a particular tissue site or body. The flow channels provided in the polymer layer may be an inherent characteristic of its material composition. Additionally, the flow channels may be chemically, mechanically, or otherwise formed in the polymer layer prior to or after manufacture of the polymer layer.

Regardless of whether cells, pores, voids, apertures, or some other combination thereof are used to define the flow channels 110, 310, 514, and 708, the porosity of the polymer layer may be different than that of an adjacent layer of biomolecules that has been applied to the polymer layer. The porosity of the polymer layer may be controlled by limiting the size of the pores, voids, and/or apertures, or by controlling the number (i.e. density) of pores, voids, and/or apertures disposed in a particular layer of material.

Certain pores, voids, and/or apertures of the layers of material may be “closed” that are not fluidly connected to adjacent cells. These closed pores, voids, and/or apertures of the layers of material may be selectively combined with pores, voids, and/or apertures of the polymer layer to prevent transmission of fluids through selected portions of the polymer layers 102, 302, 402, 502, and 702.

The polymer layers 102, 302, 402, 502, and 702 promote new tissue growth and accept in-growth of new tissue from the tissue site, tissue site, and/or wound body. The polymer layers 102, 302, 402, 502, and 702 preferably are porous and capable of accepting and/or integrating new tissue growth into the biomolecule dressings.

In any of the previous embodiments, an outside membrane layer may be used to protect the most outward layer of material from being contaminated prior to use. In one aspect, the outer membrane layer may be affixed or adhered to the biomolecule dressings such that it is easily removed by a user prior to placing it adjacent or in contact with a tissue site.

The dimensions of the polymer layers 102, 302, 402, 502, and 702 may be any size, thickness, surface area, or volume necessary to fit a desired application. In one aspect, the general shapes of the polymer layers may be formed in sheets having desired thicknesses for an application. The polymer layers may further be manufactured or formed in large sheets that may span large tissue masses and subsequently hold them in place.

In general, the polymer layers 102, 302, 402, 502, and 702 have a thickness of from about 1 mm to about 100 mm. The thickness of the polymer layers is measured in a direction normal to the tissue site or wound body. The dimensions of the polymer layers in a plane normal to the thickness dimension may vary depending on the size of the tissue site or wound body. The polymer layers may be provided in a large size and then trimmed or formed to fit the tissue site or wound body.

The pore size of the polymer layers 102, 302, 402, 502, and 702 is preferably from about 50 microns to about 600 microns. In another embodiment, the pore size of the polymer layers may be from about 250 microns to about 400 microns. Preferably, the pore size of the polymer layers may be about 100 microns, or thinner.

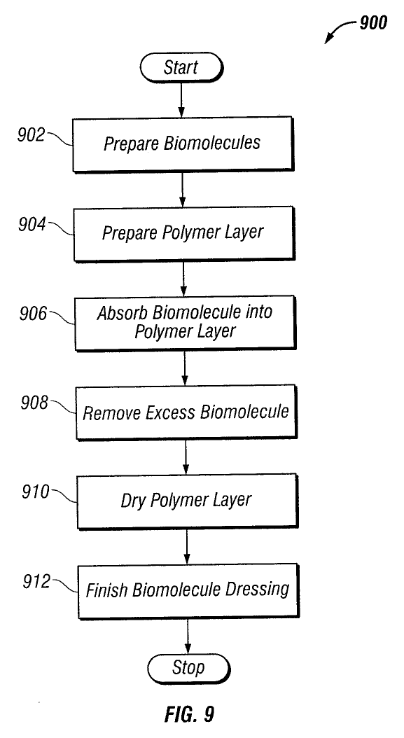

In addition to the aforementioned aspects and embodiments of the biomolecule dressing, another embodiment of the invention may include methods for coating a polymer layer, partially or completely, with biomolecules. Referring to FIG. 9, a method 900 for coating a biomolecule dressing according to an exemplary embodiment of the invention is provided. In one embodiment, the method 900 enables a biomolecule dressing to be cut, severed, or shaped in any direction and still have exposed surfaces that are coated with the biomolecules sufficient to provide the benefits described herein.

In step 902, a biomolecule is prepared and placed or stored in an appropriate vessel. Preferably, light, agitation, temperature, pressure, and other conditions are considered when storing the biomolecule. In step 904, a polymer layer is prepared and cut to a desirable size. In step 906, the polymer layer is placed in the vessel and the biomolecule is absorbed and/or adsorbed onto and through the polymer layer. This step may further comprise soaking or squeezing the polymer layer. In step 908, excess solution of the biomolecule is removed from the polymer layer. Roller nips of similar devices may be utilized to control the amount of solution removed from the polymer layer. In step 910, the polymer composition may be dried and/or weighed to determine the amount of biomolecule deposited on the polymer composition. Drying may take place in a conventional oven or other drying apparatus to a predetermined temperature and time. In step 912, the finished biomolecule dressing may be shaped, formed, trimmed, cut, or the like to complete its final shape. Additionally, in step 912, any additional manufacturing steps, such as finishing, sterilization, packaging, and the like are performed.

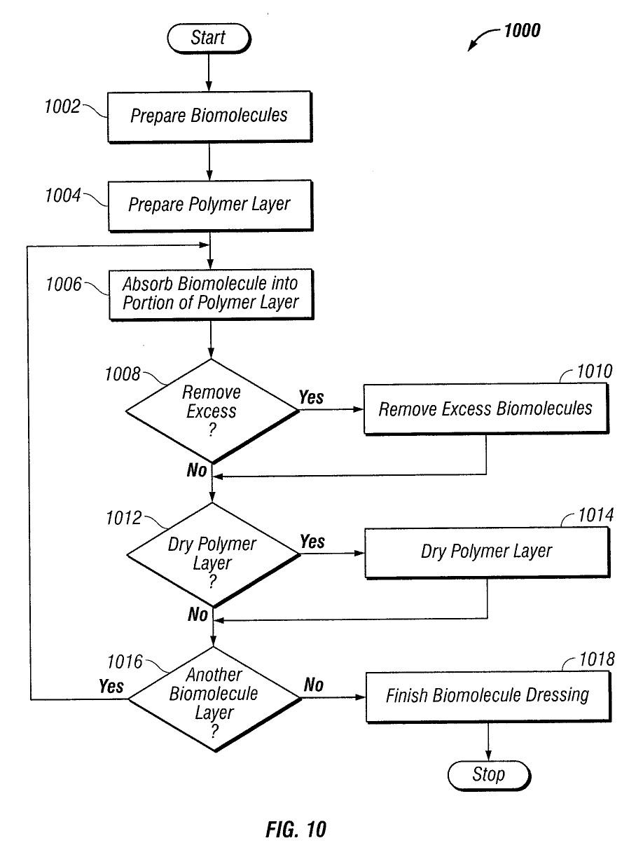

FIG. 10 illustrates an embodiment of a flow chart of another exemplary process 1000 for coating a biomolecule dressing. In step 1002, one or more biomolecules are prepared and placed or stored in separate appropriate vessels. Light, agitation, temperature, pressure, and other conditions are considered when storing the biomolecules. In step 1004, a polymer layer is prepared and cut to a desirable size. In step 1006, the polymer layer is partially dipped in the vessel containing a first biomolecule that is absorbed and/or adsorbed onto and through a first portion of the polymer layer. In this step only a portion of the polymer layer is absorbed with or adsorbed with the biomolecule. This step may further comprise soaking or squeezing the polymer layer to better absorb the biomolecules into the polymer layer.

In step 1008, an inquiry is made as to whether excess biomolecules are to be removed from the polymer layer. If the answer to this inquiry is “yes,” then in step 1010 excess solution of the biomolecules are removed from the polymer layer. This step may actually occur after each individual deposition step. Roller nips of similar devices may be utilized to control the amount of solution removed from the polymer layer.

If the answer to the inquiry at step 1008 is “no,” then in step 1012 a further inquiry is made as to whether the polymer layer may be dried. If the answer to this inquiry is “yes,” then in step 1014 the polymer layer is dried and/or weighed to determine the amount of biomolecule deposited on the polymer composition. Drying may take place in a conventional oven or other drying apparatus to a predetermined temperature and time. If the answer to the inquiry at step 1012 is “no,” then in step 1016 a further inquiry is made as to whether another biomolecule layer is to be deposited on another portion of the biomolecule layer. If the answer to this inquiry is “yes,” then another layer of biomolecules is absorbed and/or adsorbed onto and through an additional portion of the polymer layer. If the answer to the inquiry is “no,” then in step 1018 polymer layer is finished into a biomolecule dressing. At this step the polymer layer are finished into a biomolecule dressing. At this step, the biomolecule dressing may be shaped, formed, trimmed, cut, or the like to complete its final shape. Additionally, in step 1018, any additional manufacturing steps, such as finishing, sterilization, packaging, and the like may be performed.

It should be apparent from the foregoing that an invention having significant advantages has been provided. While the invention is shown in only a few of its forms, it is not just limited but is susceptible to various changes and modifications without departing from the spirit thereof.

We claim:

1. A reduced pressure dressing coated with biomolecules comprising:
a polymer material layer; and
at least one biomolecule selected from the group consisting of a hemostatic agent, an antioxidant agent, and a nitric oxide promoter, said at least one biomolecule absorbed into a portion of said polymer material layer.

2. The reduced pressure dressing coated with biomolecules of claim 1 further comprising:
additional layers of a biomolecule selected from the group consisting of a hemostatic agent, an antioxidant agent, and a nitric oxide promoter, said at least one biomolecule applied to the outer surface of said polymer material layer.

3. The reduced pressure dressing coated with biomolecules of claim 2, further comprising:
an inert layer substantially interspersed between said polymer layer and said additional layers of biomolecules layers.

4. The reduced pressure dressing coated with biomolecules of claim 1, wherein said polymer material layer is a bioresorbable material selected from the group consisting of polylactide (“PLA”) (both L-lactide and D,L-lactide), copolymer of Poly(L-lactide-co-D,L-lactide), polyglycolic acid (“PGA”), alpha esters, saturated esters, unsaturated esters, orthoesters, carbonates, anhydrides, ethers, amides, saccharides, polyesters, polycarbonates, polycaprolactone (“PCL”), polytrimethylene carbonate (“PTMC”), polydioxanone (“PDO”), polyhydroxybutyrate, polyhydroxyvalerate, polydioxanone, polyorthoesters, polyphosphazenes, polyurethanes, collagen, hyaluronic acid, chitosan; polymers incorporating one or more of hydroxyapatite, coralline apatite, calcium phosphate, calcium sulfate, calcium sulfate, calcium carbonate, carbonates, bioglass, allografts, autografts; and mixtures and/or co-polymers of these compounds.

5. The reduced pressure dressing coated with biomolecules of claim 1, wherein said first layer of material and said second layer of material have a thickness of from about 1 mm to about 100 mm.

6. The reduced pressure dressing coated with biomolecules of claim 1, wherein said polymer layer has pore sizes from about 250 microns to about 600 microns.

7. The reduced pressure dressing coated with biomolecules of claim 1, wherein said hemostatic agent is selected from the group consisting of poly-N-acetyl-glucosamine, thrombin, fibrinogen, or fibrin constituted in an aqueous solution of a non-acidic, water-soluble or water-swellable polymer, including but not limited to methyl cellulose, hydroxyalkyl cellulose, keratin sulfate, water-soluble chitosan, N-acetyllactosamine synthase, salts of carboxymethyl carboxyethyl cellulose, chitin, salts of hyaluronic acid, alginate, propylene glycol alginate, glycogen, dextran, carrageenans, chitosan, starch, amylose, and the aldehyde-oxidized derivatives thereof.

8. The reduced pressure dressing coated with biomolecules of claim 1, wherein said antioxidant agent is selected from the group consisting of glutathione, lipoic acid, vitamin E, ascorbic acid, trolox, tocopherols, and tocotrienols.

9. The reduced pressure dressing coated with biomolecules of claim 1, wherein said nitric oxide promoter is selected from the group consisting of nitric oxide, nitric oxide donor compounds, nitric oxide precursor compounds, upregulators of nitric oxide compounds, L-arginine, nitric oxide synthase, and nitroprusside.

10. A reduced pressure treatment system for applying a reduced pressure treatment to a tissue site comprising:
a polymer material layer;
at least one biomolecule selected from the group consisting of a hemostatic agent, an antioxidant agent, and a nitric oxide promoter, said at least one biomolecule absorbed into a portion of said polymer material layer;
a manifold layer located substantially over said polymer layer in communication with said tissue site; and
a reduced pressure delivery tube fluidly connected to said manifold layer to deliver reduced pressure to said tissue site.

11. The reduced pressure delivery system of claim 10, further comprising:
additional layers of material selected from one of said layer of bioresorbable microspheres and said layer of bioresorbable fibers located adjacent to one of said first layer of material and said second layer of material.

12. The reduced pressure delivery system of claim 10, wherein said polymer material layer is selected from the group consisting of polyurethane, cellulose, carboxylated butadiene-styrene rubber, polyester foams, hydrophilic epoxy foams, polyacrylate, GranuFoam®, and WhiteFoam™.

13. The reduced pressure delivery system of claim 10, wherein said polymer material layer is selected from the group consisting of bioresorbable material may be made from polylactide (“PLA”) (both L-lactide and D,L-lactide), copolymer of Poly(L-lactide-co-D,L-lactide), polyglycolic acid (“PGA”), alpha esters, saturated esters, unsaturated esters, orthoesters, carbonates, anhydrides, ethers, amides, saccharides, polyesters, polycarbonates, polycaprolactone (“PCL”), polytrimethylene carbonate (“PTMC”), polydioxanone (“PDO”), polyhydroxybutyrate, polyhydroxyvalerate, polydioxanone, polyorthoesters, polyphosphazenes, polyurethanes, collagen, hyaluronic acid, chitosan; polymers incorporating one or more of hydroxyapatite, coralline apatite, calcium phosphate, calcium sulfate, calcium sulfate, calcium carbonate, carbonates, bioglass, allografts, autografts; and mixtures and/or co-polymers of these compounds.

14. The reduced pressure delivery system of claim 10, wherein said polymer material layer is chemically modified to provide a covalent bond with said at least one biomolecules.

15. The reduced pressure delivery system of claim 10, wherein said polymer material layer is chemically modified to provide an ionic bond with said at least one biomolecules.

16. The reduced pressure delivery system of claim 10, further comprising:
at least two biomolecules, a first of said at least two biomolecules absorbed into a first portion of said polymer layer and a second of said biomolecules absorbed into a second portion of said polymer layer.

17. The reduced pressure delivery system of claim 10, wherein said hemostatic agent is selected from the group consisting of poly-N-acetyl-glucosamine, thrombin, fibrinogen, or fibrin constituted in an aqueous solution of a non-acidic, water-soluble or water-swellable polymer, including but not limited to methyl cellulose, hydroxyalkyl cellulose, keratin sulfate, water-soluble chitosan, N-acetyllactosamine synthase, salts of carboxymethyl carboxyethyl cellulose, chitin, salts of hyaluronic acid, alginate, propylene glycol alginate, glycogen, dextran, carrageenans, chitosan, starch, amylose, and the aldehyde-oxidized derivatives thereof.

18. The reduced pressure delivery system of claim 10, wherein said antioxidant agent is selected from the group consisting of glutathione, lipoic acid, vitamin E, ascorbic acid, trolox, tocopherols, and tocotrienols.

19. The reduced pressure delivery system of claim 10, wherein said nitric oxide promoter is selected from the group consisting of nitric oxide, nitric oxide donor compounds, nitric oxide precursor compounds, upregulators of nitric oxide compounds, L-arginine, nitric oxide synthase, and nitroprusside.

20. A process for making a reduced pressure dressing coated with biomolecules comprising:
preparing at least one biomolecules selected from the group consisting of a hemostatic agent, an antioxidant agent, and a nitric oxide promoter;
preparing a polymer material layer;
absorbing said at least one biomolecules on a first portion of said polymer material layer; and
finishing said reduced pressure dressing coated with biomolecules.

21. The process for making a reduced pressure dressing coated with biomolecules of claim 20, further comprising:
removing excess of said at least one biomolecules from said polymer layer.

22. The process for making a reduced pressure dressing coated with biomolecules of claim 20, further comprising:
drying said at least one biomolecules absorbed in said polymer layer.

23. The process for making a reduced pressure dressing coated with biomolecules of claim 20, further comprising:
absorbing another of said at least one biomolecules on a second portion of said polymer material layer.

24. The process for making a reduced pressure dressing coated with biomolecules of claim 20, wherein said finishing said reduced pressure dressing coated with biomolecules comprises:
processing said reduced pressure dressing coated with biomolecules by at least one of shaping, trimming, cutting, forming, sterilizing, and packaging.
